(19) United States
(12) Patent Application Publication
(10) Pub. No.: US 2010/0247623 A1
(43) Pub. Date: Sep. 30, 2010

(54) Cancer Stem Cell Vaccine

(76) Inventor: Jean-Claude Bystryn, New York, NY (US)

Correspondence Address: COOPER & DUNHAM, LLP, 30 Rockefeller Plaza, 20th Floor, NEW YORK, NY, 10112, US

(21) Appl. No.: 12/711,699

(22) Filed: Feb. 24, 2010

Related U.S. Application Data
(60) Provisional application No. 61/155,244, filed on Feb. 25, 2009.

(51) Int. Cl. A61K 39/00; A61K 9/127; A61P 35/00

(52) U.S. Cl. 424/450; 424/277.1

(57) ABSTRACT

The invention provides a method of making a vaccine for treating or preventing cancer in a patient or individual which comprises preparing the vaccine from cancer stem cells, from fractions, extracts, shed material or purified antigens derived from these cells, or from DNA or RNA coding for cancer stem cell associated antigens. In one embodiment of this invention, cancer stem cells are separated from unrelated cancer cell populations and then expended and cultured under conditions in which the cancer stem cells shed or release tumor antigens, and using the shed material or cancer stem cell antigens isolated from it as the vaccine. The invention further provides a vaccine and methods of treatment using the vaccine for any cancer is disclosed. Also disclosed are specific applications of this method to prepare such vaccines from antigens released or shed by cancer stem cells, and a particular application to make a shed, cancer stem cell, vaccine for breast cancer.

### CROSS-REFERENCE TO RELATED APPLICATION

This application claims priority based upon U.S. Provisional Patent Application Ser. No. 61/155,244, filed Feb. 25, 2009, the contents of which are incorporated by reference herein.

### FIELD OF THE INVENTION

This invention relates to cancer vaccines and more particularly to a cancer vaccine made from antigens expressed by cancer stem cells, and methods for making and using such a vaccine.

### BACKGROUND OF THE INVENTION

The invention is intended to more closely satisfy the critical and fundamental requirement that to be effective cancer vaccines must contain tumor antigens that are expressed by the cancer cells that one wishes to attack. The invention is based on recent advances in understanding the biology of cancer that point to a more effective way of doing so. The invention provides a new strategy to design and construct more effective vaccines for cancer. These advances also suggest that current cancer vaccines include many ineffective tumor antigens, limiting the effectiveness of the vaccine.

It has recently been discovered that cancerous tumors are made up of two different and unequal populations of cancer cells-stem cells, which are rare, and daughter cells, which are much more common. Stem cells comprise only a few percent of the cancer cells in a tumor, but they appear to account for the ability of the cancer to grow. They are the cells that must be destroyed for that growth to be stopped. By contrast, the daughter cells account for nearly all of the cells in a cancer, but do not have the ability to spread the tumor. These differences are readily demonstrated by injecting these two different populations of cancer cells into mice. Using techniques known in the art, it is possible to separate stem cells from daughter cells in a cancerous tumor—the first accounting for as little as one percent of the cells, the other the remaining 99%. When equal numbers of these two cell populations are injected into mice, the stem cells can induce tumors in all mice, whereas the daughter cells do not induce any tumors.

These two cell populations differ not only in their ability to induce tumors, but also in their functional and antigenic properties. For example, stem cells (but not daughter cells) have molecular machinery that renders them resistant to chemotherapy and radiotherapy, making them resistant to usual cancer treatments. Thus, while these treatments may kill the bulk of cells in a cancer, causing the tumor to shrink temporarily, the stem cells seem unaffected, survive, and cause the tumor to regrow. The better way to treat cancer would be to attack and kill the cancer stem cells. The disclosure described here is intended to permit cancer vaccines to be constructed in a manner that will allow them to attack and destroy this cell population more effectively.

There is another difference between stem and daughter cancer cells that is relevant to cancer vaccines. The antigens expressed by these two types of cells also differ. For example, cancer stem cells but not daughter cells can express the antigens CD44 high and CD24 low. While these differences have been observed with a limited number of antigens, they, however, establish the principle that the antigenic properties of the two cell types differ. This difference is critical to the design of more effective cancer vaccines that target cancer stem cells. Cancer vaccines work by containing, and thus stimulating immune responses against, antigens expressed by the tumor cells it is wished to attack. Thus, cancer vaccines, to be most effective should contain antigens expressed by cancer stem cells.

### SUMMARY OF THE INVENTION

The invention provides a method of making a cancer vaccine comprising: obtaining a population of cancer cells from one more tumor samples; separating cancer stem cells from other cells, culturing the cancer stem cells in culture media under conditions wherein the cancer stem cells will shed or release cancer stem cell antigens into the culture media; and separating the shed or released cancer stem cell antigens from the cancer cell stem cells to obtain partially purified cancer stem cell antigens.

The invention further provides a method of making a cancer vaccine where the vaccine is prepared from an inactivated cancer stem cell, a purified antigen associated with a cancer stem cell phenotype, or a DNA or RNA molecule coding for such an antigen.

The invention also provides a stem cell cancer vaccine, wherein the vaccine is made from purified, individual tumor antigens or fragments thereof expressed by cancer stem cells, from DNAs or RNAs coding for cancer stem cell antigens, from partially purified fractions or extracts of cancer stem cells that contain cancer stem cell antigens, from material released or shed by cancer stem cells, from killed or inactivated whole cancer stem cells, or from viruses transfected with DNA or RNA coding for cancer stem cell antigens to express these antigens.

### DETAILED DESCRIPTION OF THE PREFERRED EMBODIMENTS

The invention provides a method of making a cancer vaccine comprising obtaining a population of cancer cells from one more tumor samples; separating cancer stem cells from other cells, culturing the cancer stem cells in culture media under conditions wherein the cancer stem cells will shed or release cancer stem cell antigens into the culture media; and separating the shed or released cancer stem cell antigens from the cancer cell stem cells to obtain partially purified cancer stem cell antigens. The vaccine may be prepared from multiple inactivated cancer stem cell lines from multiple purified antigens associated with a cancer stem cell phenotype, or from multiple DNAs or RNAs coding for such antigens. The vaccine may also be prepared from an inactivated cancer stem cell, a purified antigen associated with a cancer stem cell phenotype, or a DNA or RNA molecule coding for such an antigen.

The invention further provides a stem cell cancer vaccine, wherein the vaccine is made from purified, individual tumor antigens or fragments thereof expressed by cancer stem cells, from DNAs or RNAs coding for cancer stem cell antigens, from partially purified fractions or extracts of cancer stem cells that contain cancer stem cell antigens, from material released or shed by cancer stem cells, from killed or inactivated whole cancer stem cells, or from viruses transfected with DNA or RNA coding for cancer stem cell antigens to express these antigens.

The stem cell vaccine may be made from multiple different lines of cancer stem cells, and may be prepared from different lines of cancer stem cells derived from different types of cancers, such that a single vaccine is effective against different types of cancers. In addition, antigens obtained from the different lines of cancer may be combined into a single vaccine that is effective against different types of cancers.

In a further embodiment, the vaccine is prepared from the material released or shed into culture medium by one or more lines of cancer stem cells, and contains a broad spectrum of different tumor antigens, is partially purified, is enriched with antigens that are expressed on the external surface of the cancer cells, and is free of toxic cellular and nuclear material. The invention additionally provides a cancer stem cell derived vaccine in a pharmaceutically effective vehicle, with or without an adjuvant.

The vaccine may treat lung, breast, prostate, colon, stomach, ovary, brain, or skin cancer. In one embodiment, the vaccine is a stem cell breast cancer vaccine made from antigens shed or released from human breast cancer stem cells, made from the human breast cancer cell lines MB.HB-11, MB.HB-13, MB.HB-15, and MB.HB-16 or from antigens shed or extracted from cell lines or from DNA or RNA obtained from these cells lines.

The vaccine may be administered together with an adjuvant that increases the frequency, strength, and/or duration of vaccine induced immune responses. The adjuvant may include encapsulating the vaccine into liposomes together with at least one cytokine that can upregulate antigen induced immune responses; admixing the vaccine together with a TLR ligand that can upregulate antigen induced immune responses such as TLR-7, TLR-8 or TLR-9 or other ligands alone or in combination; combining the vaccine encapsulated into liposomes together with at least one cytokine and with TLR ligands; and admixing with adjuvants such as montainide. The cytokine may be IL-2, GM-CSF, or a combination thereof.

The invention also provides a method for treating cancer in a patient in need of such treatment comprising administering an effective amount of a stem cell cancer vaccine in a pharmaceutically effective vehicle, which may additionally comprises an adjuvant effective to increase the frequency, strength, or duration of vaccine induced immune response.

Administration can be performed, for example, intravenously, orally, nasally, via implant, transmucosally, transdermally, intramuscularly, and subcutaneously. The following delivery systems, which employ a number of routinely used pharmaceutical carriers, are only representative of the many embodiments envisioned for administering the instant compositions:

Injectable drug delivery systems include solutions, suspensions, gels, microspheres and polymeric injectables, and can comprise excipients such as solubility-altering agents (e.g., ethanol, propylene glycol, and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials), and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols, and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, xanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue, Roche Molecular Systems, Inc., Branchburg, N.J., USA).

The core of the invention disclosed herein is a method wherein the cancer vaccines are constructed so as to contain tumor antigens expressed by cancer stem cells. This is a general method that can be applied to make vaccines against any cancer. It differs from current methods used to make cancer vaccines in that these are usually made from antigens expressed by the predominant type of cells in the cancer—which are daughter cells. Thus, current vaccines may not contain sufficient amounts of the antigens expressed by cancer stem cells. This deficiency may account for the limited effectiveness of current cancer vaccines.

In one sense, the invention is an extension of the construction of cancer vaccines made from shed antigens which is intended to improve the clinical effectiveness thereof by enabling the vaccine to better target the subpopulation of cells in a cancer that seems most responsible for its growth and spread. As with the shed antigen vaccine technology, this improvement can be applied to improve the effectiveness of shed antigen vaccines against any cancer. More broadly, this invention can be applied to improve the effectiveness of any cancer vaccine constructed using any other technology.

Isolation and identification of cancer stem cells: The present invention is based on constructing a vaccine from cancer stem cells, fractions, or extracts of these cells, tumor antigen(s) known to be expressed by cancer stem cells, or DNA or RNA coding for cancer stem cell antigens. Cancer stem cells can be isolated and identified using procedures known to those familiar with the art.

One procedure isolates individual cells in fresh, surgically resected, tumor tissue by treating the tissue with enzymes or by mechanically mincing it finely. The isolated cells can then be cloned by limiting dilution or other standard technique, and each clone tested for its ability to induce tumors in nude mice. Those clones that induce tumors are presumed to be cancer stem cells and can be expended and maintained in master and working cell banks to provide uniform cell populations for further analysis and use. An alternate procedure can be used with those cancers in which specific cancer stem cell antigens are known. Tumor cells isolated as described above can be treated with specific labeled antibodies that react with the stem cell markers and the stained cells separated from the bulk of unstained cells and collected by flow cytometry or other procedures.

Identification of the isolated cells as stem cells can be confirmed by using a number of established techniques. Beside determining their ability to induce tumors in nude mice, these include as an example examining their functional activity (stem cells have a side population profile; i.e. the ability to exclude certain dyes or small molecules) or antigenic phenotype (expressing antigens associated with stem cells). The technology involved in identifying and collecting cancer stem cells is rapidly evolving, and other procedures may be used to identify and collect these cells.

These cells, or the antigens they express, can then be used to make cancer vaccines as described below:

Procedure to make cancer stem cell vaccines: To practice this invention, cancer vaccines containing stem cell antigens can be made using several approaches known in the art. They include constructing the vaccine from:
1. Single or multiple purified tumor antigens which have been shown to be expressed by cancer stem cells and preferably have been shown to stimulate anti-tumor immune responses in humans;
2. DNA or RNA which code for these antigens;
3. Partially purified fractions or extracts of stem cells, preferably ones which are enriched in cancer stem cell antigens;
4. Whole cancer stem cells which have been inactivated by irradiation or other maneuver;
5. Antigens present in the material that is shed or released by, or otherwise obtained from, cancer stem cells;
6. Viruses that have been transfected to express the cancer stem cell antigen(s) of interest; and
7. Any of the other methods currently used to make cancer vaccines, in which the antigen(s) used are cancer stem cell antigen(s).

A particularly desirable embodiment of this invention is to construct cancer stem cell vaccines from several different lines of cancer stem cells selected on the basis that they express different patterns of cancer stem cell antigens and/or from multiple purified antigens derived from these cells, or that are released or shed by each of these cell lines. These applications result in a very broadly polyvalent vaccine (a vaccine that contains many different antigens). This embodiment is intended to minimize several problems inherent in the construction of any cancer vaccine. These requirements include the following which are required for a cancer vaccine to be effective:

a) The vaccine must contain antigens expressed by the specific cancer it is wished to treat. This is difficult to know, as the pattern of tumor antigens expressed by the same type of cancer in different individuals is variable, as is the pattern of tumor antigens expressed by different tumor nodules in the same individual and by different tumor cells in the same nodule. Furthermore, whatever these antigens are they can change during tumor progression in the same person.

b) These antigens must be able to stimulate clinically effective anti-tumor immune responses, but very little is known about the identity of such antigens at present.

Because of this heterogeneity in the expression of antigens expressed by different tumors and tumor cells within the same tumor as well as in the functional properties of the immune responses induced by these antigens; cancer stem cell vaccines constructed from a single stem cell antigen or cell line may lack the cancer stem cell antigens expressed by the tumor to be treated and/or antigens able to stimulate clinically effective anti-tumor immune responses.

Both problems are minimized by constructing polyvalent vaccines that contain a broad spectrum of different tumor antigens that are expressed by cancer stem cells. The larger the number of antigens in a cancer vaccine the greater its chances of containing antigens expressed by cancer stem cells in the tumor to be treated and that some of these antigens will be able to stimulate clinically effective anti-stem cell immune responses.

As with any cancer vaccines, the effectiveness of vaccines prepared from cancer stem cells, their extracts or their antigens can be enhanced by combining or administering the vaccine with an appropriate adjuvant.

Cancer stem cell vaccines made from shed antigens. A preferred embodiment of this invention is to construct polyvalent cancer stem cell vaccines from the material released or shed into culture medium by several different lines of cancer stem cells.

Preparing the vaccine from shed material as opposed to whole tumor cells or other types of extracts is advantageous because: shed material contains a wide range of tumor antigens, permitting the construction of a broadly polyvalent vaccine;
a) these antigens are particularly relevant for vaccine therapy, as they are enriched in antigens expressed on the external surface of the tumor cells, where they can be seen by and interact with host immune defense mechanism;
b) the shed antigens are partially purified as they are separated from the bulk of cytoplasmic and nuclear material that is released much more slowly; and
c) being depleted of cytoplasmic and nuclear material the vaccine is less likely to contain material that can cause toxicity or interfere with the activity of the vaccine than vaccines prepared from whole cells or their extracts.

Preparing the vaccine from shed material as opposed to individual purified antigens or DNA or RNA that code for such antigens is advantageous because the vaccine contains many more tumor antigens. Hence it is much more likely to contain antigens expressed by the tumor to be treated and able to stimulate protective immune responses—absolute requirements for a vaccine to be effective. Unfortunately, a limited number of cancer stem cell antigens have been identified and little is known about their ability to induce protective immune responses. Thus, the ability of vaccines made from a single or limited number of antigens to be clinically effective is uncertain.

In another embodiment, the spectrum of cancer stem cell antigens in the vaccines can be further increased by selecting the cancer stem cell lines that are used on the basis that their antigenic phenotype differs, i.e. they express different patterns of cancer antigens. The type of cancers targeted by the vaccine can also be increased by preparing it from stem cell derived from tumors of different histological types.

In a further improvement, the vaccine can be prepared from cancer stem cells that have been adapted to long term growth in serum-free medium to exclude potentially harmful serum components from the vaccine.

Preparation of a shed, breast cancer stem cell vaccine. In another embodiment of this invention, the methods described above can be applied to make a breast cancer stem cell vaccine from the material that is released or shed by different lines of breast cancer stem cells. In a specific application of this embodiment, four breast cancer stem cell lines were isolated and cloned as described above. These four cell lines are denominated as MB.HB-11, MB.HB-13, MB.HB-15, MB.HB-16. All four lines were confirmed to be stem cell-like based on their ability to induce tumors in nude mice, having a side population profile, and expressing antigens associated with cancer stem cells. An additional criterion used to select these four cell lines is that they express different patterns of tumor associated antigens. Alternate lines of breast cancer stem cells can be used if available, and preferably should express different and complimentary patterns of breast cancer tumor antigens.

The four lines of breast cancer stem cells used to produce the vaccine were isolated from a resected primary nodule of breast cancer. Histologically, the tumor was an invasive ductal carcinoma. By immunohistochemistry, the cells were 100% estrogen receptor (+), 100% progesterone receptor (+), 100% Erb-2/neu (+), 5% p53 (+) and 80% p27 (+). The tumor tissue was rinsed in culture medium, dissected into small pieces with scissors, digested for 1 hr with an enzyme mixture and dispase, washed, plated on tissue culture plates coated with collagen type 1 and grown in Ham's F12 medium supplemented with 10% FBS, penicillin, streptomycin, and L-glutamine. The cells were then grown in T-75 culture flasks in a humidified incubator at 37 C with 5% carbon dioxide. Once the cells grew continuously, the concentration of FBS was gradually reduced in steps every 4 weeks until the cells were fully adapted to long-term growth in serum-free medium supplemented with transferrin, insulin, hydrocortisone, triiodothyronine, β-estradiol, and progesterone).

The cells were then cloned by limiting dilutions using conventional procedures and adapted to long term growth in serum-free medium. Histologically, the cells in all clones had a malignant phenotype with a fairly homogenous “plasmacytoid” appearance. The cloned cells all expressed the breast cancer epithelial specific antigen (ESA) as well as at least one other antigenic marker associated with breast cancer including Her-2/neu, MAGE-1, MAGE-3, CEA or others. Four clones were selected based on their ability to induce tumors in nude mice and on expressing different pattern of breast cancer associated antigens, as illustrated in Table 1. The expression of different pattern of tumor antigens by these cells indicates their expression of stem cell related antigens may be equally diverse, increasing the variety of breast cancer stem cell antigens in the vaccine.

TABLE I
Antigen Phenotype of Breast Cancer Cell Lines

| | Breast Cancer Stem Cell Lines | | | |
|---|---|---|---|---|
| Antigen | MB.HB-11 | MB.HB-13 | MB.HB-15 | MB.HB-16 |
| NK1-C3 | + | + | + | + |
| MAGE-1 | − | − | na | + |
| MAGE-3 | + | + | + | + |
| NY-ESO-987 | + | − | + | − |

A vaccine was prepared from the material shed in 3 hrs by these cell lines using procedures previously disclosed in for example, U.S. Pat. No. 5,030,621, which is incorporated herein by reference.

What is claimed is:

1. A method of making a cancer vaccine comprising: obtaining a population of cancer cells from one more tumor samples; separating cancer stem cells from other cells, culturing the cancer stem cells in culture media under conditions wherein the cancer stem cells will shed or release cancer stem cell antigens into the culture media; and separating the shed or released cancer stem cell antigens from the cancer cell stem cells to obtain partially purified cancer stem cell antigens.

2. A method of making a cancer vaccine according to claim 1 wherein the vaccine is prepared from an inactivated cancer stem cell, a purified antigen associated with a cancer stem cell phenotype, or a DNA or RNA molecule coding for such an antigen.

3. A method of making a cancer vaccine according to claim 1, wherein the vaccine is prepared from multiple inactivated cancer stem cell lines from multiple purified antigens associated with a cancer stem cell phenotype, or from multiple DNAs or RNAs coding for such antigens.

4. A stem cell cancer vaccine, wherein the vaccine is made from purified, individual tumor antigens or fragments thereof expressed by cancer stem cells, from DNAs or RNAs coding for cancer stem cell antigens, from partially purified fractions or extracts of cancer stem cells that contain cancer stem cell antigens, from material released or shed by cancer stem cells, from killed or inactivated whole cancer stem cells, or from viruses transfected with DNA or RNA coding for cancer stem cell antigens to express these antigens.

5. A method according to claim 1, wherein the stem cell vaccine is made from multiple different lines of cancer stem cells.

6. A method according to claim 1, wherein the vaccine is prepared from different lines of cancer stem cells derived from different types of cancers, and wherein a single vaccine is effective against different types of cancers.

7. A method according to claim 3, wherein the vaccine is prepared from different lines of cancer stem cells derived from different types of cancers, and wherein a single vaccine is effective against different types of cancers.

8. A method according to claim 4, wherein the vaccine is prepared from different lines of cancer stem cells derived from different types of cancers, and antigens obtained from them are combined into a single vaccine is effective against different types of cancers.

9. A method according to claim 1, wherein the vaccine is prepared from the material released or shed into culture medium by one or more lines of cancer stem cells, and wherein the vaccine contains a broad spectrum of different tumor antigens, is partially purified, is enriched with antigens that are expressed on the external surface of the cancer cells, and is free of toxic cellular and nuclear material.

10. A method according to claim 3, wherein the vaccine is prepared from the material released or shed into culture medium by one or more lines of cancer stem cells, and wherein the vaccine contains a broad spectrum of different tumor antigens, is partially purified, is enriched in antigens that are expressed on the external surface of the cancer cells, and minimizes potentially toxic cellular and nuclear material.

11. A method according to claim 1, wherein the cancer is a lung, breast, prostate, colon, stomach, ovary, brain, or skin cancer.

12. A method according to claim 9, wherein the cancer is a lung, breast, prostate, colon, stomach, ovary brain or skin cancer.

13. A method according to claim 10, wherein the vaccine is a stem cell breast cancer vaccine made from antigens shed or released from human breast cancer stem cells.

14. A method according to claim 1, wherein the vaccine is made from the human breast cancer cell lines MB.HB-11, MB.HB-13, MB.HB-15, and MB.HB-16 or from antigens shed or extracted from cell lines or from DNA or RNA obtained from these cells lines.

15. A method according to claim 13, wherein the vaccine is made from the human breast cancer cell lines MB.HB-11, MB.HB-13, MB.HB-15, and MB.HB-16 or from antigens shed or extracted from cell lines or from DNA or RNA obtained from these cells lines.

16. A method according to claim 1, wherein the vaccine is administered together with an adjuvant that increases the frequency, strength, and/or duration of vaccine induced immune responses.

17. A method according to claim 13, wherein the vaccine is administered together with an adjuvant that increases the frequency, strength, and/or duration of vaccine induced immune responses.

18. A method according to claim 14, wherein the vaccine is administered together with an adjuvant that increases the frequency, strength, and/or duration of vaccine induced immune responses.

19. A method according to claim 16, wherein the adjuvant includes encapsulating the vaccine into liposomes together with at least one cytokine that can upregulate antigen induced immune responses; admixing the vaccine together with a TLR ligand that can upregulate antigen induced immune responses such as TLR-7, TLR-8 or TLR-9 or other ligands alone or in combination; combining the vaccine encapsulated into liposomes together with at least one cytokine and with TLR ligands; and admixing with adjuvants such as montainide.

20. A method according to claim 19, wherein the at least one cytokine is IL-2, GM-CSF, or a combination thereof.

21. A method for treating cancer in a patient in need of such treatment comprising administering an effective amount of a vaccine according to claim 1 in a pharmaceutically effective vehicle.

22. A method according to claim 21, wherein the vaccine additionally comprises an adjuvant effective to increase the frequency, strength, or duration of vaccine induced immune response.

23. A cancer stem cell derived vaccine according to claim 1 in a pharmaceutically effective vehicle.
